(19) United States
(12) Patent Application Publication
(10) Pub. No.: US 2011/0060268 A1
(43) Pub. Date: Mar. 10, 2011

(54) NAIL TREATMENT DEVICE

(75) Inventors: Eyal Ben Moshe, Ramat-Gan (IL); Mordechay Moshkovich, Ra'anana (IL); Boaz Nitzan, Sde-Varburg (IL)

(73) Assignee: POWER PAPER LTD., Petah Tikva (IL)

(21) Appl. No.: 12/937,050

(22) PCT Filed: Apr. 7, 2009
(86) PCT No.: PCT/IL09/00399

§ 371 Date: Nov. 15, 2010

Related U.S. Application Data
(60) Provisional application No. 61/044,123, filed on Apr. 11, 2008.

(51) Int. Cl. A61N 1/30

(52) U.S. Cl. 604/20; 604/501

(57) ABSTRACT

Provided is a nail treatment device including an active delivery device for disposing on the nail and for promoting delivery of an active agent into the nail to treat a nail infection; and at least one means for preventing delivery of the active agent to the skin. Further provided is a method of treating a nail infection including applying onto an infected nail a nail treatment device including an active delivery device for promoting delivery of an active agent into the nail to treat a nail infection and at least one means for preventing delivery of the active agent to the skin.

### FIELD

The present invention relates to a nail treatment device. Moreover, the present invention is of a nail treatment device for active delivery of an active substance to the nail.

### BACKGROUND

A variety of infections are known to affect the nail. Nail infections include fungal infection, bacterial infection, viral infection, psoriasis infection and combinations thereof.

Onychomycosis is a fungal infection of the nail and surrounding skin. In the most common form of onychomycosis, the fungus invades the nail bed under the nail plate, beginning at the hyponychium and then migrates proximally through the underlying nail matrix. Onychomycosis causes nails to thicken, split, discolor and disfigure and may cause pain.

Psoriasis of the nails occurs in about half the people affected with psoriasis. Nail psoriasis manifests itself with pitting and deformation of the nail, thickening of the nail, onycholysis and discoloration.

It has been found by the inventors that iontophoresis promotes the delivery of active agents into the nail and provides an effective method of treating nail infections including onychomycosis.

A substance to be delivered by iontophoresis should preferably be disposed in close proximity to the delivery electrode of an iontophoresis device. In iontophoretic treatment of a nail infection, a drug in contact with the delivery electrode, which is disposed on an infected nail area may be delivered into the nail. The inventors observed that if the drug in contact with the nail is also in contact with the surrounding skin, at least some if not most of the drug may be delivered through the skin instead of through the nail. This problem appears to be unique to active delivery to the nail. Presumably, this may be a result of the typically lower resistance exhibited by skin compared to nail and because a drug which is actively delivered will typically be delivered through the current pathway of least resistance.

In order to optimize delivery of an active agent into the nail, short circuiting through the skin should be avoided.

It would therefore be advantageous to have an iontophoresis device and method of use thereof which prevents delivery of the active agent through the skin and optimizes delivery of the active agent through the nail and into the nail, such as is provided by the present invention.

### SUMMARY

Aspects of the invention include a nail treatment device for active delivery of an active substance to the nail. One aspect relates to a nail treatment device comprising an active delivery device for disposing on the nail and for promoting delivery of an active agent into the nail to treat a nail infection and at least one means for preventing delivery of the active agent to the skin. The device may be worn by a user and may deliver the active agent when mobile and/or when stationary. The device may include a skin mask. The skin mask may be integrally formed with the device or may be part of a kit comprising the skin mask and the nail treatment device. The skin mask may include a locator for accurate placement on the digit of a user. The treatment device may include a locator for disposing and/or suitably interconnecting with the skin mask locator for accurate application of the treatment device.

A further aspect relates to a nail treatment device comprising an active delivery device for disposing on the nail and for promoting delivery of an active agent into the nail to treat a nail infection and at least one means for preventing delivery of the active agent to the skin, wherein the at least one means for preventing delivery of the active agent to the skin comprises an electrode and/or formulation displacement prevention means.

An additional aspect relates to a nail treatment device comprising an active delivery device for disposing on the nail and for promoting delivery of an active agent into the nail to treat a nail infection and at least one means for preventing delivery of the active agent to the skin, wherein the at least one means for preventing delivery of the active agent to the skin comprises a means for separating the nail from the skin

In a still further aspect, the at least one means for preventing delivery of the active agent to the skin comprises an electrode and/or formulation displacement prevention means and a means for separating the nail from the skin.

Another aspect relates to a method of treating a nail infection comprising applying a nail treatment device on an infected nail, the nail treatment device comprising an active delivery device for disposing on the nail and for promoting delivery of an active agent into the nail to treat a nail infection and at least one means for preventing delivery of the active agent to the skin. In one aspect a skin mask with a locator means may be first applied onto the digit, after which the nail treatment device with a corresponding device locator may be applied over the skin mask such that the skin mask locator and the device locator correspond with each other and may be suitably interconnected and the treatment device is accurately placed on the digit.

### BRIEF DESCRIPTION OF THE DRAWINGS

The various features of the invention will best be appreciated by simultaneous reference to the description which follows and the accompanying drawings and in which:

FIG. 1 shows schematically a device for iontophoretic delivery of an active agent into the nail according to one aspect of the present invention;

FIG. 2 shows schematically a device for iontophoretic delivery of an active agent into the nail according to one aspect of the present invention;

FIG. 3 shows schematically a device for iontophoretic delivery of an active agent into a nail, wherein the device is attached to a toe according to one aspect of the present invention;

FIG. 4 shows schematically a device for iontophoretic delivery of an active agent into the nail according to one aspect of the present invention;

FIG. 5 shows schematically a nail delivery device attached to a non-big toe according to one aspect of the present invention;

FIG. 6 shows schematically a kit including a device and a skin mask according to one aspect of the present invention;

FIG. 6a shows schematically a skin and nail separating means according to one aspect of the present invention;

FIG. 6b shows a schematic cross sectional view of part of the nail delivery device on the nail according to one aspect of the present invention;

FIG. 6c shows schematically a frame disposed on a toe of a user according to one aspect of the present invention;

FIG. 7a shows a schematic cross sectional view of part of the nail delivery device on the nail according to one aspect of the present invention;

FIG. 7b shows a schematic cross sectional view of part of the nail delivery device on the nail according to an aspect of the present invention;

FIG. 7c shows a schematic cross sectional view of part of the nail delivery device on the nail according to an aspect of the present invention;

FIG. 7d shows a schematic cross sectional view of part of the nail delivery device on the nail according to an aspect of the present invention;

FIG. 7e shows a schematic cross sectional view of part of the nail delivery device on the nail according to an aspect of the present invention;

FIG. 8 shows schematically a method of use of an iontophoretic delivery device for treatment of a nail infection according to one aspect of the present invention;

FIG. 9 shows schematically a method of use of an iontophoretic delivery device for treatment of a nail infection according to one aspect of the present invention;

FIG. 10 shows graphical representation of average changes in healthy toenail growth (mm) between each visit to visit 1 in Group A (Terbinafine & Patch) and Group B (Terbinafine);

FIG. 11 shows graphical representation of frequency (N, %) of patients with distance changes above 1.5 mm from visit 1 by visit and treatment group;

FIG. 12 shows graphical representation of frequency (N, %) of patients with fungal elements in their nail specimen by visit and treatment group; and

FIG. 13 shows graphical representation of Terbinafine content (μg/cm²) in nails at visit 4 and visit 5 by treatment group.

### DETAILED DESCRIPTION

It was discovered that iontophoretic delivery of an antifungal agent into the nail is effective in the treatment of nail fungal infection such as but not limited to onychomycosis. In the context of the present application, the term “iontophoresis” means any method of electrical delivery of substances, including electrotransportation, iontophoresis, electroosmosis, electroporation, and/or a combination thereof. The term ‘antifungal’ as used herein refers to any substance or composition including same which is effective in treatment of a fungal infection. The term ‘active agent’ or ‘active substance’ as used herein refers to any pharmaceutical, drug, homeopathic agent, herb, vitamin, cosmetic or cosmeceutical. The term includes any substance which has a therapeutic, biological or aesthetic effect. The term ‘nail’ as used herein refers to a toenail or fingernail. The term may also refer to a claw, talon or animal nail equivalent.

As described above, a problem of active delivery of a substance to the nail using current when compared to active delivery of a substance to the skin is that due to the lower resistance of the skin, if the substance to be delivered or the delivery electrode is contacted with skin in addition to nail, the substance or most of the substance will be delivered to the skin and not to the nail. In order to optimize active delivery of an active substance to the nail, it is advantageous to avoid or limit contact of the main electrode and/or substance to be delivered with the skin. This problem is not encountered when the target delivery site is the skin and the formulation or the delivery electrode contacts the nail region. In addition, this problem is not characteristic of passive delivery of a drug to the nail, wherein the nail resistance and current pathway are not relevant. An additional problem, associated with contact of the main electrode with the skin, is that due to the relatively high resistance of the nail compared with the skin, a higher current density than used and tolerated on skin may be necessary in order to deliver an active substance into the nail. As such, if the electrode which is in contact with the nail also contacts the skin, the high current density may be harmful to the skin, causing burns.

Before explaining at least one embodiment of the invention in detail, it is to be understood that the invention is not limited in this application to the details of construction and the arrangement of the components set forth in the following description or illustrated in the drawings. The invention is applicable to other embodiments of being practiced or carried out in various ways. Also, it is to be understood that the phraseology and terminology employed herein is for the purpose of description and should not be regarding as limiting.

The principles and operation of a device according to the present invention may be better understood with reference to the figures. The figures show exemplary embodiments of the present invention and are not limiting.

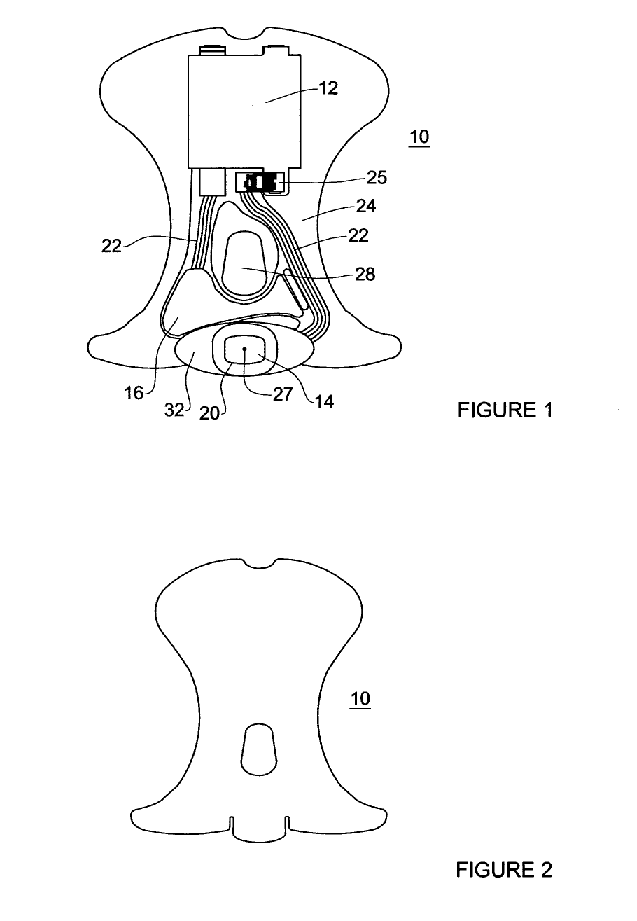

FIG. 1 shows schematically a device for active delivery of an active agent to the nail 10 according to one aspect of the present invention. The device may be an iontophoresis delivery device. As can be seen from FIG. 1, the device 10 includes at least one power source 12, at least one main electrode 14, at least one counter electrode 16 and at least one means for preventing delivery of the active agent to the skin. The device may include at least one drug reservoir 20.

The device 10 may be thin and flexible. The device 10 may be lightweight. In some aspects the device 10 may be less than about 1 mm thick. In one aspect, the device 10 may be less than about 0.5 mm thick. The device 10 may be flexible in order that it can conform to the contours of the digits and surrounding areas. In one aspect, the device 10 is for application for prolonged periods of time such as more than several hours. As such, the device 10 may be configured in order for the user to have substantially full mobility. In an alternative aspect, the device 10 may be any suitable active delivery device suitable for delivery of an active agent to the nail wherein the device further features at least one means for preventing delivery of the active agent to the skin. FIG. 2 shows schematically a device for iontophoretic delivery of an active agent to the nail according to one aspect of the present invention, wherein the device is a plaster like patch device 10.

The device 10 may be powered by any suitable power source 12. A suitable power source may include at least one galvanic couple, which may comprise the at least one main electrode 14 and the at least one counter electrode 16 and/or at least one battery which may be integral to the device 10 or may be an external component. The power source 12 may be at least one thin and flexible battery. In one aspect the power source 12 may be a non-thin and flexible battery such as but not limited to a button battery or any type of battery. The power source 12 may be of any suitable voltage. In one aspect the power source 12 may provide up to about 40V. In one aspect the power source 12 is configured to supply any suitable current density, such as a current density of up to about 1000 μA/cm². The power source 12 may be in electrical connection by any suitable means 22 with the counter electrode 16 and the main electrode 14, which are disposed in spaced apart relation on a device body 24 or device frame 24. The power source 12 may be disposed in any suitable region of the device 10. In one aspect the power source 12 may be disposed in a part of the device to be disposed on the foot area. The size of the device 10 may be configured to accommodate the size of the power source 12 used.

The device may include any additional suitable electronic circuitry 25, such as but not limited to a chip. The electronic circuitry may control the current and voltage. In one aspect the circuitry may facilitate constant current or constant voltage. In a further aspect, the circuitry may facilitate providing current which is not above a predetermined value. The device may be configured to provide any suitable current such as direct current, alternating current or pulsed current.

The main delivery electrode 14 and the counter electrode 16 may be any suitable electrode, made by any suitable technique. In one aspect, the main electrode 14 may include one or a plurality of holes 27 or other suitable openings. The hole 27 may facilitate releasing excess surplus formulation, when the device 10 is disposed on the nail. The device 10 may further includes a type of pad of absorbent material (not shown in FIG. 1) disposed between the device frame 24 and main electrode 14, which may absorb the released excess formulation. In such a way the excess formulation may be prevented from leaking from the nail to the skin. The size of the hole and the number of holes may be chosen so as not to affect the electrode function. In one aspect, the main delivery electrode 14 is for disposing on the infected nail and for delivering the active agent into the nail. The counter electrode 16 may be disposed on the device 10 such that on application onto a user it is disposed on any suitable non-nail area. In the device 10 shown in FIG. 1, the counter electrode 16 is disposed in the device 10 such that it will be disposed on the skin of the digit in the same plane as the active delivery electrode 14. In one aspect, the counter electrode 16 may be configured such that it is in the same plane in a linear arrangement with the active electrode 14 and is further disposed to at least one side of the delivery electrode 14. The size of the counter electrode 16 may be configured so that the electrode is thin and flexible and less prone to damage. In view that the device it not only wearable and configured for a user when stationary, but is also configured for full mobility of a subject, it is desirable that the electrode is sized for optimal thinness for comfort and also for the subject to be able to wear footwear such as a sock and shoe or equivalent over the device. If the counter electrode 16 is smaller, it may be bulky in order to facilitate the desired electrical requirements. Further, a counter electrode 16 which is configured around the sides as well as in a linear arrangement from the active electrode 14 facilitates optimal direction of current and multidirectional current for delivery of the active agent to a plurality of areas in the nail. In one aspect, hydrogel 26 (not shown in FIG. 1) may be disposed on the main delivery electrode 14 and the counter electrode 16.

The at least one means for preventing delivery of the active agent to the skin may comprise any suitable means for preventing contact of the main electrode 14 and formulation with the skin. The means for preventing contact of the main electrode 14 and formulation with the skin may include a movement electrode and/or formulation displacement prevention means 28, for preventing displacement of the main electrode 14 and/or formulation on movement of the toe or foot. The means for preventing contact of the main electrode 14 and formulation with the skin may in addition feature a means for preventing contact of the formulation with the skin when pressure is applied to the affected region. The means for preventing delivery of the active agent to the skin may be configured such that the means does not hinder or prevent delivery of the active agent into the nail.

The device 10 may be configured so that when applied to a user, the user is able to be mobile or move the treated area. The device 10 may be of any suitable size, and on application of the device 10 on the nail, toe and foot regions of a user, the device may be disposed on the joints of the toes used in movement and walking. It was observed that a delivery device, which is configured so that it is disposed on the part of the toe and/or foot which is flexed on movement, resulted in pulling of the main electrode and/or formulation downwards, when the toe or foot was flexed or moved. This problem was especially prevalent with the big toe. A result of such movement was that the formulation was displaced and undesirably contacted with the surrounding skin area resulting in delivery of the active agent into the skin instead of the nail. One non limiting example of a suitable means for preventing displacement of the main electrode and/or formulation is a device aperture 28 or suitable opening in the device frame 24, which on application of the device 10 on a user is disposed about the toe movement joint, such as the first metatarsal phalange joint of the toe. The device aperture 28 provides a solution to the movement displacement problem, resulting in a user being able to flex or move the foot and/or toe joint without putting strain on the device 10 and preventing displacement of the main electrode or formulation from the nail to the skin. The device aperture 28 may be sized and positioned in order that it does not interfere with the desired function of the device.

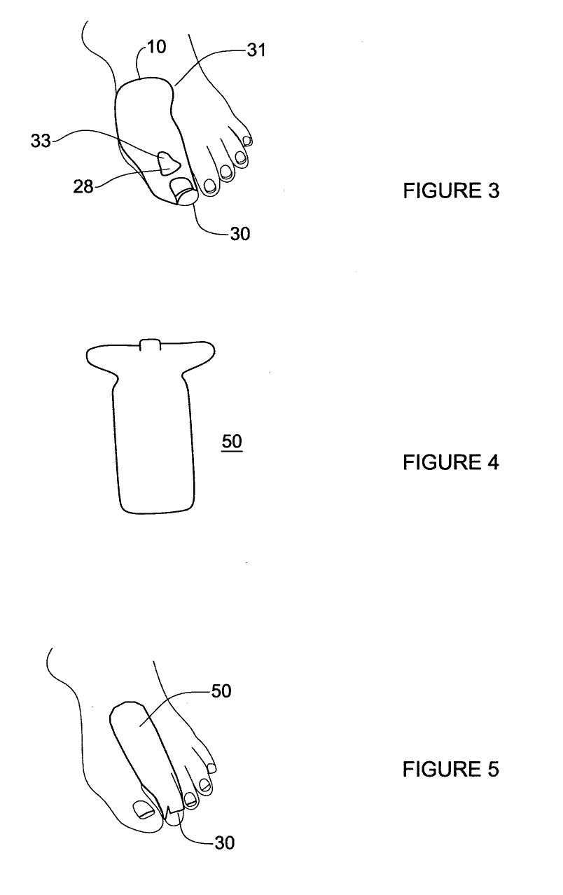

FIG. 3 shows schematically a device for iontophoretic delivery of an active agent into the nail 10 such as shown in FIG. 2, wherein the device 10 is attached to a toe 30 and the surrounding foot area 31 of a user. The device 10 shown in FIG. 3 is attached to the big toe 30. As can be seen from FIG. 3 the device may include a device aperture 28, which may be disposed in device 10, such that when the device is applied to a user, the device aperture 28 will be disposed about the joint 33 of the toe 30.

Other suitable means for enabling movement of the foot whilst ensuring that the delivery electrode and formulation are not displaced and contacted with the skin include use of elastic and stretchable material in at least the region of the device disposed on the toe foot joint. In an aspect wherein the device 10 includes stretchable material, the stretchable material may preferably not be placed on regions of the device where it may interfere with the desired function of the device, such as in the region of the main electrode or in any region of the device such that it will promote delivery of the formulation into the skin.

In an alternative aspect, the device 10 may be constructed such that the device is not disposed over the toe or foot movement joints. In such an aspect, the device may not need a movement electrode and/or formulation displacement prevention means 28.

The joints in the smaller toes are not moved to the same extent as the big toe joint and therefore an aperture movement electrode/formulation displacement prevention means as described hereinabove may not be essential in the devices for treatment of the nails of the smaller toes. FIG. 4 shows schematically a device 50 for iontophoretic delivery of an active agent to the nail according to a further aspect of the present invention. As can be seen from FIG. 4, the plaster like device 50 does not include an aperture electrode/formulation displacement prevention means for enabling movement of the foot as shown in the device of FIGS. 1, 2 and 3. The device 50 of FIG. 4 is intended for application on one of the non-big, smaller toes. The other components of the device 50 are substantially as described for 10 hereinabove and below. The shape of the device 50 for the smaller toes may differ according to the ergonometry of the relevant foot area. FIG. 5 shows the device 50 attached to the second toe.

One suitable means for preventing contact of the main electrode and/or formulation with the skin may feature at least one skin mask 32. The skin mask 32 allows contact of the main electrode and/or formulation with the nail. As can be seen in FIG. 1, device 10 may include a skin mask 32. A skin mask 32 may be any suitable means or separator for separating and/or preventing contact of the skin from the nail. In one aspect the skin mask may be disposed to cover and mask at least some of the skin surrounding a nail. In one aspect the skin mask or any alternative separator means may be disposed to mask some or all the skin surrounding the nail, but in such a way as to enable electrical contact between the power source and the counter electrode which may be disposed on the skin. The suitable means for separating and/or preventing contact of the skin with the nail may be disposed such that on use it would not be applied to the area of the toe where the counter electrode is to be contacted as this may prevent closing of the circuit with the counter electrode. In some embodiments, the skin mask will be disposed on the nail perimeter and the skin. Non-limiting examples of a suitable separator means or other means for separating and/or preventing contact of the skin with the nail include masking tape, a nail frame, nail varnish and combinations thereof. In one non-limiting example non-conductive nail varnish may be applied around the skin of the nail. The suitable separator or other means for separating and/or preventing contact of the skin with the nail may be constructed from a non-conductive material, which is substantially impermeable to the active agent formulation.

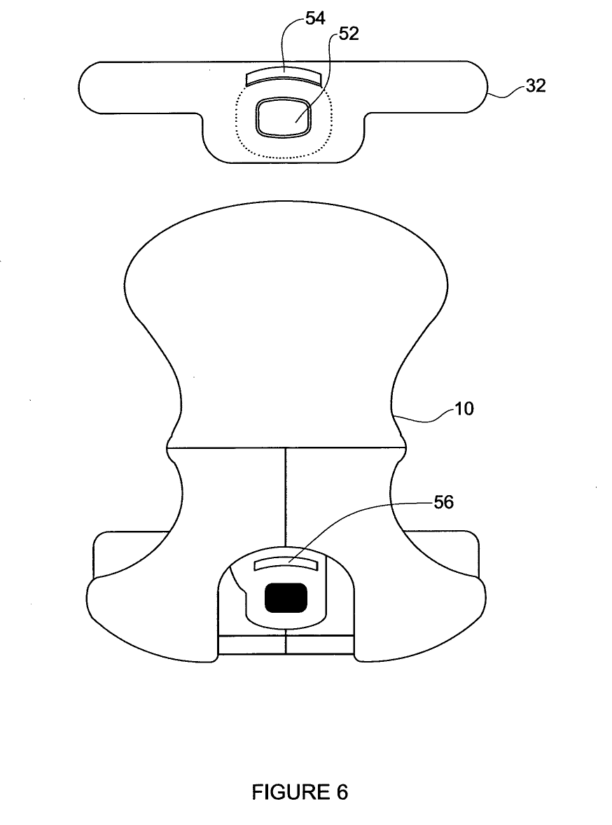

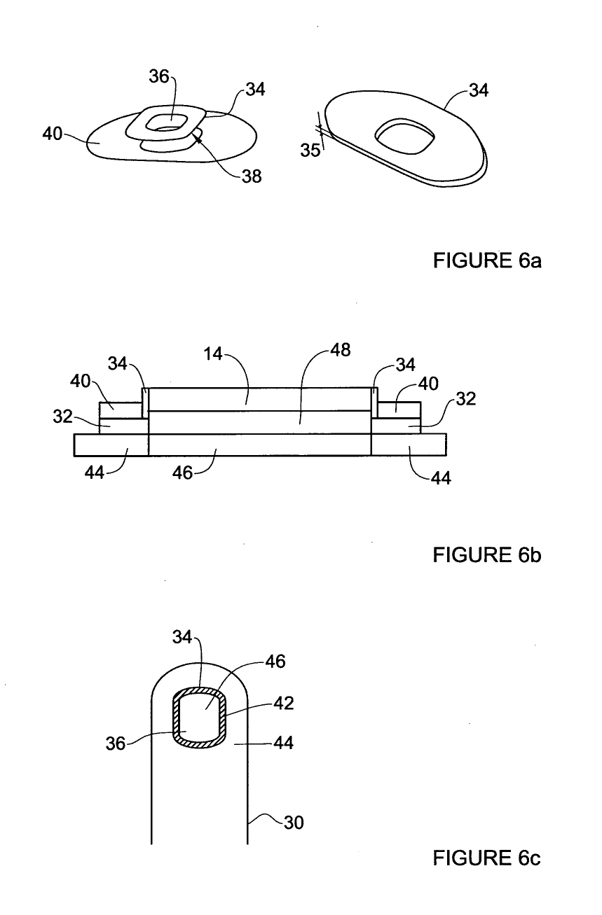

The skin mask or other suitable separator means may be an integral part of the device as shown in FIG. 1. Alternatively, the skin mask may be part of a kit as shown in FIG. 6. In an aspect wherein the skin mask is part of a kit, such as shown in FIG. 6, the skin mask 32 may be applied to the digit by a user before application of the device 10. The skin mask 32 may include an aperture 52 which is suitable for exposing the nail part of the digit. The skin mask may include any suitable attachment means to attach to the skin, such as adhesive. The adhesive underside of the skin mask 32 may be protected before use by a liner (not shown in FIG. 6). The skin mask may include a locator 54 and/or other suitable markings such that a user may correctly apply the skin mask 32 onto the toe. In one embodiment the locator 54 may be for placing on the perimeter of the nail and skin below the lunala, such as on the eponychium. The locator 54 may be a raised portion (a male part of the locator) of the mask. The skin mask 32 may be placed such that the raised portion 54 points upwards away from the digit. The device 10 may include a corresponding locator 56, for corresponding and fitting with the skin mask locator 54, for example an insert or aperture into which the raised portion of the skin mask locator 52 may fit. The type of locator is not intended to be limiting and any complimentary suitable locators may be used in the skin mask 32 and the device 10. Use of a skin mask 32 as part of a kit may be advantageous, as it facilitates covering skin regions of the digit before application of the formulation. When the skin mask 32 is an integral part of the device 10, the formulation may for any number of reasons contact the underside of the skin mask, before the device 10 has been applied to the user. Such contact would negate the function of the skin mask 32. Use of a skin mask 32 with a locator 54 is advantageous as it aids a user to correctly apply the skin mask 32 and the device 10 to the nail and the digit. The skin mask locator 54 enables correct positioning of the skin mask 32 for optimal covering of the skin, while facilitating an uncovered nail region. The skin mask locator 54 further enables correct positioning of the device 10 on top of the mask 32 such that the electrodes and formulation are correctly disposed on the nail region. If the skin mask 32 or the device 10 is not applied accurately, the active electrode and/or the formulation may be placed such that they are disposed on the skin. Such incorrect positioning may prevent the device 10 from producing the desired therapeutic effect. Accurate positioning is therefore very important, however the small size of the nails on the digits prevents such precise positioning without the use of a suitable locator means.

FIG. 1 shows the device comprising a drug reservoir 20. The suitable means for separating the skin from the nail may be integrated with the drug reservoir 20. In an alternative aspect, the suitable means for separating and/or masking the skin from the nail may not be part of the drug reservoir 20.

The drug reservoir 20 may be an integral part of the device 10, such that the drug reservoir 20 may be built in to the device 10 or the device 10 may be supplied with the drug reservoir 20 attached in any suitable way to the device 10. In one aspect the drug reservoir 20 is supplied separately from the device 10 and may be attached by a user to the device 10 or the nail before using the device 10. The drug reservoir 20 may be disposed on the main delivery electrode 14. The drug reservoir 20 may be disposed on a hydrogel 26 which is disposed on the main delivery electrode.

FIG. 6a shows schematically a skin and nail separating means according to one aspect of the present invention. The skin and nail separating means may be configured as a drug reservoir 20. The reservoir 20 may include a nail frame 34 which may be in a ring conformation with a central nail frame aperture 36. In some aspects, the nail frame 34 may not be part of the drug reservoir 20. A skin and/or nail contacting surface 38 of the nail frame 34 may include any suitable adhesive for adhering the nail frame 34 to the area surrounding the nail to be treated. The nail frame 34 may be a means for framing or separating the skin from the nail in order to prevent the formulation and/or delivery electrode from contacting the skin. The nail frame 34 may be constructed from a non-conductive material, which is substantially impermeable to the active agent formulation. In one aspect, the nail frame 34 is made from polystyrene type foam. The nail frame 34 may be of any suitable dimensions such that the formulation can not seep under and/or over the sides of the nail frame 34 and such that the nail treatment area is not covered by the nail frame 34. In one aspect the height 35 of the nail frame 34 is less than about 2 mm. In some aspects the height 35 is less than about 1 mm. The volume capacity of the nail frame aperture 36 may be up to about 5 cm³. In one aspect the volume capacity may be up to about 1 cm³. The nail frame 34 may include or have attached thereto tape 40 disposed on the sides of the nail frame 34 and/or around the nail frame 34. In one aspect, the nail frame may be disposed on tape 40. When the nail frame is disposed on tape 40, it enables more facile removal of the device. The tape 40 may function in place of mask 32 described in FIG. 1. The tape 40 may be adhesive tape and may be one sided adhesive tape. In an aspect wherein the tape 40 functions instead of mask 32, the tape 40 may be applied such that it covers and masks the skin on either side of the nail and may also function to further anchor the nail frame 34 around the nail. Alternatively, as shown in FIG. 6b the tape 40 may function in addition to mask 32. In the event of formulation 48 leakage, tape 40 which is suitable for disposing about mask 32 may restrict the leaked formulation between the mask 32 and tape 40 and prevent the formulation from contacting any of the device components. The masking tape 40 may be non-conductive and may be impermeable to the drug formulation.

FIG. 6c shows schematically the nail frame 34 disposed on a digit 30. The device 10 or 50 may be disposed on the patient, such that the nail frame 34 is in contact with only the nail part 42 of the nail perimeter and the nail frame aperture 36 is disposed on the nail. The main delivery electrode and the active agent may be disposed within the nail frame aperture 36. Optionally, in some aspects, the device 10 or 50 may be disposed on a user, such that the nail frame 34 is in contact with only the skin 44 surrounding the nail perimeter, or the nail frame 34 may be disposed such that it contacts part of the surrounding skin 44 around the nail and part of the nail part 42 of the nail perimeter. The position of the nail frame 34 on the digit 30 and nail 46 is very important and needs to be applied with precision. The device 10 and 50 may include markings and locator means on the non-body contacting surface of the device to enable easy correct application of the device including the nail frame 34.

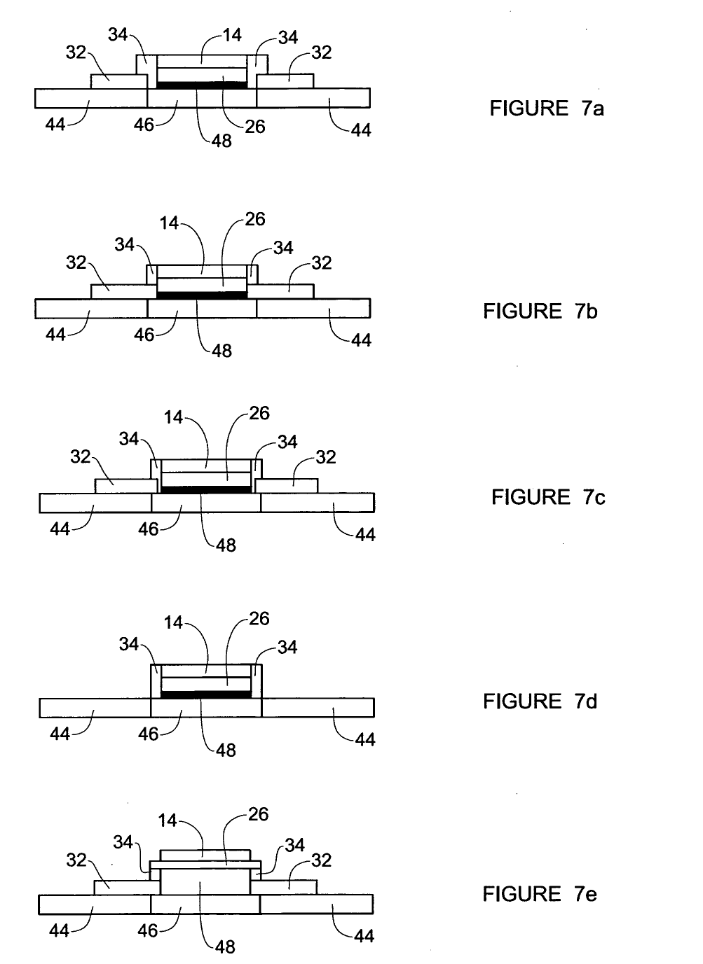

FIG. 7a shows a schematic cross sectional view of part of the nail delivery device on the nail according to one aspect of the present invention. From FIG. 7a, it can be seen that a nail frame 34 may be disposed on the nail 46 about a hydrogel 26, wherein the hydrogel may be disposed on the main electrode 14. The hydrogel 26 may be configured to absorb the applied formulation 48 and prevent the formulation 48 from contacting the skin area 44. A mask 32 may be disposed at the sides of the nail frame 34, such that the mask 32 covers at least part of the skin 44 and separates the skin from the nail 46.

FIG. 7b shows a schematic cross sectional view of part of the nail delivery device on the nail according to an alternative aspect of the present invention. From FIG. 7b, it can be seen that the nail frame 34 is disposed on the mask 32, such that the mask contacts the nail perimeter.

FIG. 7c shows a schematic cross sectional view of part of the nail delivery device on the nail according to a further alternative aspect of the present invention. From FIG. 7c, it can be seen that the part of the nail frame 34 is disposed on the mask 32, such that part of the nail frame 34 and part of the mask 32 contact the nail perimeter.

FIG. 7d shows a schematic cross sectional view of part of the nail delivery device on the nail according to a further alternative aspect of the present invention. From FIG. 7d, it can be seen that the device includes a nail frame 34, but does not include an additional mask 32.

FIG. 7e shows a schematic cross sectional view of the part of the nail delivery device on the nail according to a still further aspect of the present invention. From FIG. 7e, it can be seen that a hydrogel 26 is disposed in such a way that it is disposed on the nail frame 34. The hydrogel may be of a size such that it may be longer than the main electrode 14 or nail frame aperture. Part of the hydrogel which is disposed on the nail frame may not contact the nail or formulation. In such a way, the hydrogel 26 may be configured as a type of cover or cap to further prevent the formulation from leaking over the sides of the nail frame.

The formulation may be applied to the infected nail. Care must be taken that the formulation is not contacted with the skin. The formulation may be a gel, which may be applied accurately to the nail area, without contacting the surrounding skin area. Alternatively, the formulation may be preapplied to the device or may be applied to the device by a user, such that it is disposed within the nail frame.

In an aspect wherein the drug reservoir or formulation is supplied separately by a user, the invention provides a kit featuring the device and the separate formulation and/or reservoir and/or skin mask.

In one aspect the present invention provides a method of treating a nail infection. The terms ‘treatment’ ‘treat’ and ‘treating’ as used herein encompass any treatment of a nail infection, such as onychomycosis or psoriasis and includes: preventing the infection or disease from occurring in a subject which may be predisposed to the disease; inhibiting the infection or disease, i.e. arresting its development; and/or relieving the disease, i.e. causing regression of the disease. Relieving the disease means attaining improvement in the subject's condition, including, but not limited to clinical improvement, microbiological improvement and aesthetic improvement.

Non-limiting examples of active agents used to treat onychomycosis and which may be used with the present invention include terbinafine, naftifine, ketoconazole, fluconazole, itraconazole, terconazole, natamycin, nyastin, amphotericin B, 5-Fluorocytosine, bifonazole, chlortrimazole, econazole, fenticonazole, miconazole, oxiconazole, tolnaftate, ciclopirox, undecylenic acid, sulbentine and amorlifine in free base, ionic form, salt form or a derivative thereof.

Non-limiting examples of active agents used to treat psoriasis and which may be delivered to the nail with the device of the present invention include clobetasol, salicylic acid, tazarotene, calcipotriene, methotrexate, acitretin, cyclosporine, ibuprofen, naproxen, sulfasalazine, fluocinonide and triamcinolone in free base, ionic form, salt form or a derivative thereof.

The nail infection, such as onychomycosis or psoriasis may be treated by applying an iontophoresis device as described herein to the infected nail area. The device may include the formulation. In an aspect, wherein the formulation is not attached to the device, the formulation may be applied to the main electrode of the device or applied directly to the nail region to be treated. In one aspect, the formulation may be applied to a hydrogel disposed on the main electrode. After the formulation has been applied, (in an aspect where the device does not include the formulation), the device may be applied to the nail and foot area. The device is applied by ensuring that the nail frame as previously described is disposed on the digit and that the nail frame covers the perimeter of the nail. Slight pressure on the nail frame may adhere the nail frame to the area of the toe. The nail frame may include any additional adhesive means to ensure the correct positioning of the nail frame and formulation. The device may include adhesive means in order for the user to adhere the device to the surrounding skin. In a device for use with the big toe, wherein the device is to be disposed over a movement joint of the toe and/or foot, the means about the movement joints, such as a device aperture may be correctly positioned by a user. In some aspects, the device includes wings or other attachment means which can be disposed and adhered around the toe and foot. In an aspect wherein a skin mask is not integral with the device, the skin mask may be applied and adhered to the digit, such that the skin mask locator may be positioned on the eponychium. The mask may be adhered to the digit by adhesive or other attachment means on the device frame. In an aspect, wherein the formulation is not preapplied, the formulation may be applied to the reservoir of the device or to the nail. The device may then be applied to the digit, such that the device locator interlocks or equivalent thereof with the skin mask locator and the reservoir and main electrode are positioned on the nail.

In some aspects, the contact of the device with the body area causes closing of the circuit of the device with the nail and the current promotes delivery of the active agent from the formulation onto and into the nail for treatment of the nail.

The device may be removed from the body area at the end of the device application time. Time of application and frequency of treatment may vary and may be according to a treatment regimen.

In some aspects a pretreatment can be applied prior to use of the device. Non-limiting examples of pretreatments include applying a cleanser, applying a moisturizing composition, cutting nail, removing dry skin, hair removal, bathing, softening treatment, applying an anti-irritant, applying a permeation enhancer, heating, microporation, electrical stimulation, applying a formulation comprising a pharmaceutically active ingredient, applying a formulation comprising a cosmetically active ingredient or a combination thereof.

In some aspects a post treatment can be applied to the body area after application of the device. Non-limiting examples of post treatments include applying an occlusion formulation, applying a cleanser, cooling, applying a nail varnish, applying a formulation comprising a pharmaceutically active ingredient, applying a formulation comprising a cosmetically active ingredient or a combination thereof.

Use of the present invention can facilitate alleviation and elimination of the nail infection. Duration of effect can be affected by time and frequency of application, type and amount of current used, severity of condition and inactivation of the active agent delivered and present in the nail and matrix. The duration of the effect of the treatment may vary. Repeated use may have a synergistic effect on duration and extent of treatment result.

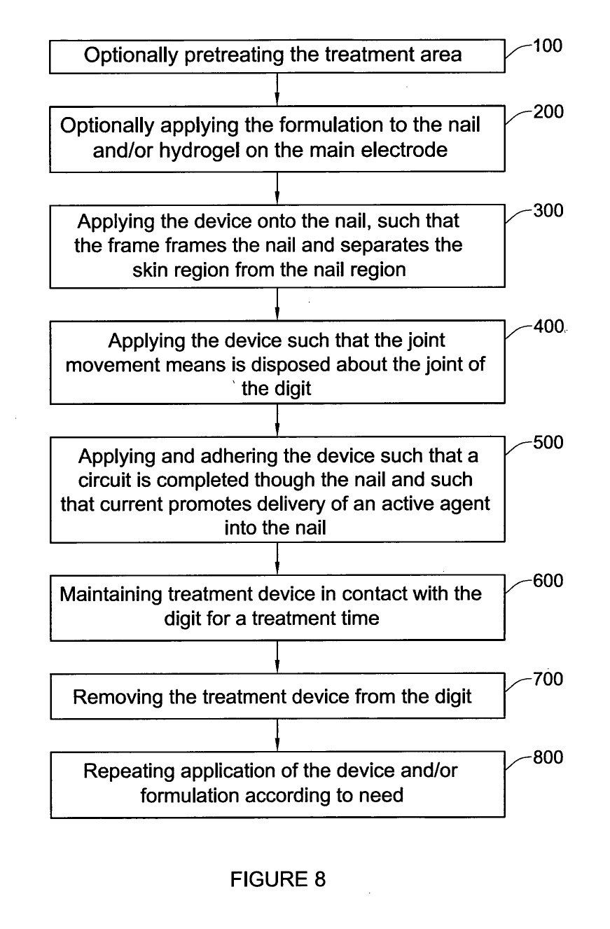

FIG. 8 shows schematically a typical treatment according to the present invention. The treatment area may be optionally pretreated (100) as described hereinabove. A device such as 10 or 50 or as described hereinabove may be provided. In an aspect, wherein the device does not include the formulation, the formulation may be applied onto the main electrode or onto an optional hydrogel disposed on the main electrode of the device and/or the formulation may be applied to the treatment area of the nail (200). The device may then be applied to the treatment area of the nail and/or surrounding skin area, such that a device nail frame is disposed around the perimeter of the nail to frame the nail and separate the skin surrounding region from the nail region and wherein the nail frame aperture is not disposed on the skin (300). At least one additional means for masking the skin from the nail may be applied, such as a masking tape for applying to the skin surrounding the nail and/or nail varnish. The device may be applied such that a digit or toe joint movement means if incorporated in the device is disposed about the movement joint of the digit, such as the toe (400). The device may be applied and adhered to the toe and foot area, such that the active electrode is disposed on the nail and the counter electrode is contacted with the skin and a circuit is completed through the nail, so that current promotes delivery of an active agent to the nail (500). The treatment device is contacted with the digit for a treatment time (600). The device and/or formulation may be removed from the treatment area after the treatment time (700). The treatment area may be optionally treated with a post treatment as described hereinabove. The same device, a different device or a different sample of the same device may again be applied and the treatment may be repeated at a suitable time interval (800). The treatment and the application of the device and/or formulation may be repeated according to need.

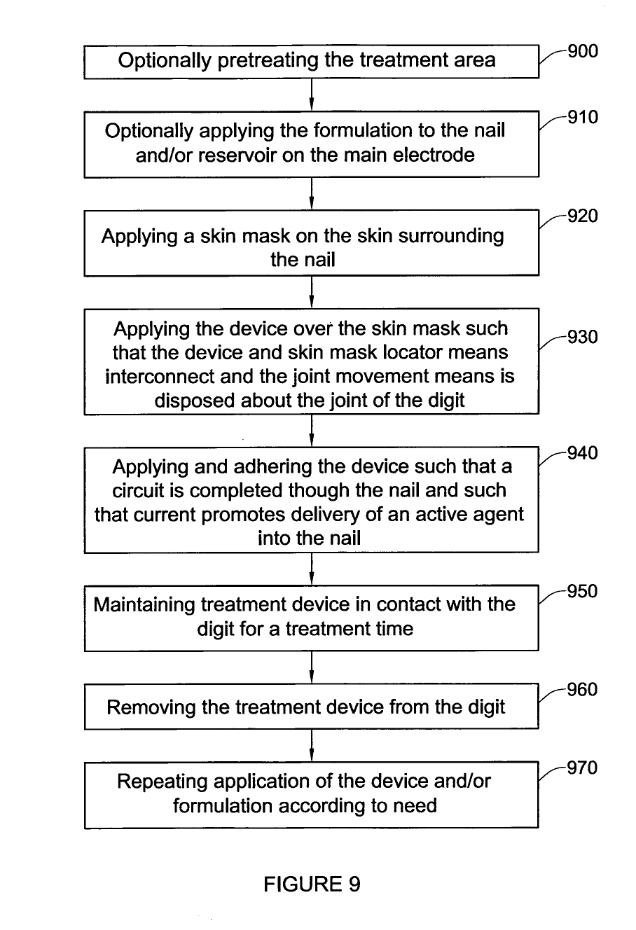

FIG. 9 shows schematically a treatment according to an aspect of the present invention. The treatment area may be optionally pretreated (900) as described hereinabove. A device such as 10 or 50 or as described hereinabove may be provided. In an aspect, wherein the device does not include the formulation, the formulation may be applied to the device reservoir and/or the formulation may be applied to the treatment area of the nail (910). A skin mask may be applied and adhered to the skin surrounding the nail, such that the nail region is exposed. The skin mask may be applied such that a locator on the skin mask is disposed about a suitable place on the digit, such as the eponychium (920). The device may then be applied over the skin mask. The device may be positioned such that a locator on the device fits into or onto the locator of the skin mask. The device may be applied such that a digit or toe joint movement means if incorporated in the device is disposed about the movement joint of the digit, such as the toe (930). The device may be applied and adhered to the toe and foot area, such that the active electrode is disposed on the nail and the counter electrode is contacted with the skin and a circuit is completed through the nail, so that current promotes delivery of an active agent to the nail (940). The treatment device is contacted with the digit for a treatment time (950). The device and/or formulation may be removed from the treatment area after the treatment time (960). The treatment area may be optionally treated with a post treatment as described hereinabove. The same device, a different device or a different sample of the same device may again be applied and the treatment may be repeated at a suitable time interval (970). The treatment and the application of the device and/or formulation may be repeated according to need.

In one aspect, the treatment may be configured for home use or any other non-supervised use. In other aspects, the treatment may be conducted in a supervised environment.

The device of the present invention is configured for optimal delivery of an active agent by iontophoresis to the nail and for preventing delivery of the active agent to the skin facilitating improved treatment of the nail infection.

### Example 1

A clinical trial was conducted to evaluate delivery of terbinafine HCl with an iontophoretic device according to an aspect of the present invention in patients with toenail onychomycosis. 38 patients suffering from distal and lateral subungual toenail onychomycosis were enrolled into the study and were divided randomly into group A (20 subjects) and group B (18 subjects). Onychomycosis was confirmed by both KOH examination and mycological culture, wherein samples were taken from the distal parts of the nail. Nail samples were divided, one part being used for direct KOH examination and the second part for fungal culture using Sabouraud's Dextrose agar (Novamed, Jerusalem, Israel). The identification of the fungus was done on the basis of morphologic characteristics.

Group A was tested with 1% terbinafine HCl and an iontophoretic patch using a constant current density of 100 μA/cm². Group B was tested with 1% terbinafine HCl without iontophoresis. Treatment period was overnight wear, every day, five days per week, for 4 weeks. During the treatment period visits were conducted every 10 days (visits 2-4). The follow-up period was 10 weeks from the end of treatment (visit 5). At each visit nail photography and KOH examination were performed. Routine blood test; complete blood count and liver functions tests were performed at the before study initiation, at the end of the treatment and at the end of the follow-up period. Terbinafine content in affected nail samples was determined by clipping nails at visits 4 & 5. Nails were then weighed and cut into symmetric shapes in order to estimate their area. Symmetric shapes of nails were then cut into small pieces and inserted into tubes with 0.5 ml DDW. Subsequently, methanol 0.5 ml was added and tubes were sonicated. The eluted solution was analysed by HPLC.

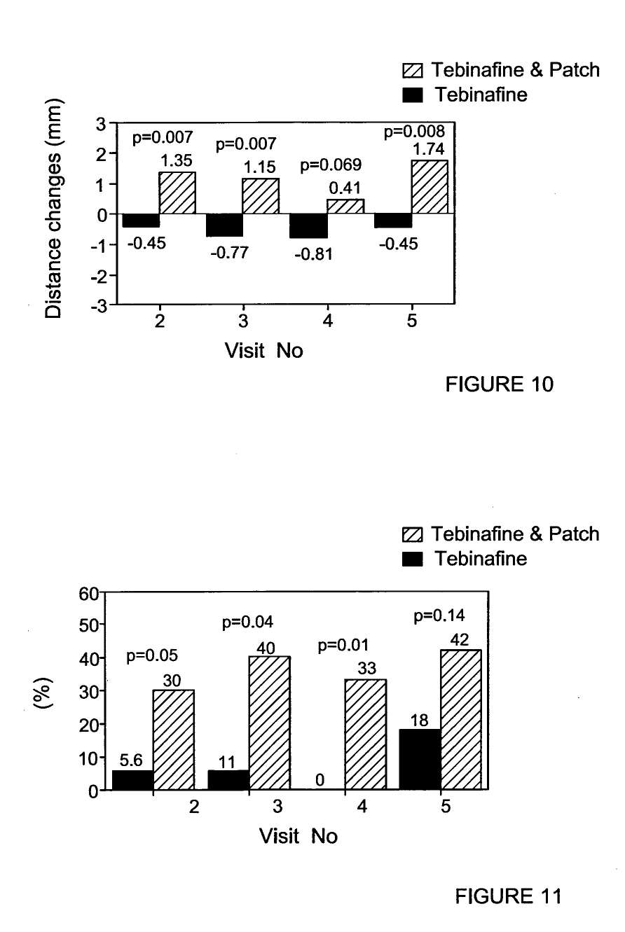

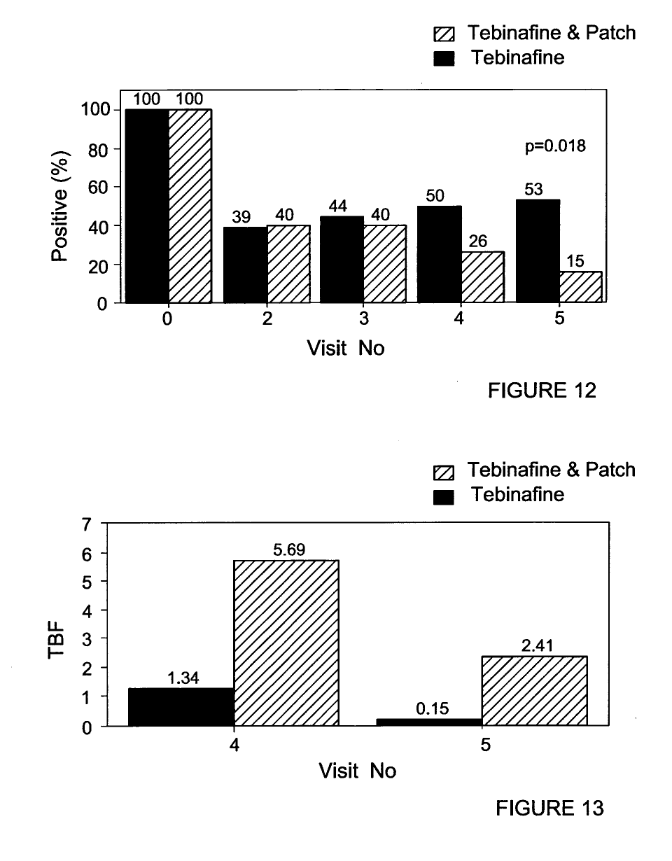

A significant clinical response was recorded in group A patients treated with terbinafine under a constant current density of 100 μA/cm². Patients from the active group A showed significant growth of healthy toenails (FIGS. 10 and 11). The percentage of patients having healthy toenail growth above 1.5 mm at the end of treatment in group A was 40% compared to 11% in patients treated with terbinafine without current (passive group B). A significant mycological improvement was observed during and after treatment in patients in Group A, (FIG. 12) whereby the percentage of patients having fungal elements (KOH) in nail specimens decreased significantly 10 weeks following the completion of treatment (15% in the active group A versus 53% in the passive group B).

Terbinafine (TBF) nail content was much higher at visit 4 among patients in the active group A (5.69 μg/cm³) than in patients from the passive group B (1.34 μg/cm²). Statistically significant differences were observed in mean values of terbinafine content in nail samples between treatment groups at visit 5 (p<0.05) (FIG. 13). At the end of the follow up period terbinafine nail content in the active group A was found to be 2.41 μg/cm², compared to 0.15 μg/cm² in the passive group B. It was noted that the terbinafine content in Group A even at the end of the follow up period was considerably higher than terbinafine's minimal inhibition concentration (MIC) against dermatophytes (0.1 to 1.0 μg/cm²).

The device of the present invention appears to deliver terbinafine in clinically effective amounts into the nail and is efficacious, safe and well tolerated for the treatment of nail onychomycosis.

### Example 2

A nail treatment device without means for preventing delivery of the active agent to the skin was applied onto the big toes of a group of about 10 subjects. The subjects were allowed to move and walk about. The majority of the subjects suffered from burns on the skin, resulting from contact of high current density with the skin. The active electrode and/or formulation were found to have been displaced from the nail and to have contacted the skin of these subjects. A nail treatment device including means for preventing delivery of the active agent to the skin, such as a skin mask and an electrode and/or formulation movement displacement prevention means according to an aspect of the present invention was also tested on the big toes of a group of about 10 subjects. It was observed that none of the subjects suffered from burns or hot spots, indicating that the main electrode and formulation did not contact the skin.

### Example 3

A nail treatment device was supplied to a group of subjects to self apply to the big toe. It was observed that each subject applied the device in a different way with non-accurate placement of the active electrode and formulation on the digit. The group of subjects was then give a nail treatment device with locator means according to an aspect of the present invention and it was observed that all the subjects applied the device and mask correctly, such that the active electrode and formulation were disposed on the nail and not on the skin of the digit.

One skilled in the art can appreciate from the foregoing description that the broad devices and techniques of the aspects of the present invention can be implemented in a variety of forms. Therefore, while the aspects of this invention have been described in connection with particular examples thereof, the true scope of the aspects of the invention should not be so limited since other modifications will become apparent to the skilled practitioner upon a study of the specification, and following claims.

1.-19. (canceled)

20. A kit for treatment of a nail condition, the kit comprising:
a skin mask for covering skin regions surrounding the nail of a digit and separating the skin from the nail, the skin mask comprising:
a non-conductive material mask suitable to be disposed about a nail and mask the skin surrounding the nail;
an aperture in the mask suitable for exposing the nail part of the digit;
attachment means for attachment of the skin mask to the skin; and
a locator on the skin mask suitable for accurate placement of the skin mask on the digit of a user, such that the skin mask is suitable to cover the skin and facilitate an uncovered nail region; and
a nail treatment device comprising:
at least one main active electrode;
at least one counter electrode; and
a nail treatment device locator corresponding to the skin mask locator, for interlocking with the skin mask locator suitable for accurate placement of the treatment device on the digit;
wherein the skin mask locator facilitates placement of the nail treatment device on top of the skin mask, such that the at least one main active electrode is suitable for placement on the nail region.

21. The kit of claim 20, further comprising a formulation and wherein the skin mask locator facilitates positioning of the nail treatment device on top of the skin mask, such that the formulation is disposed on the nail region.

22. The kit of claim 20, wherein the skin mask locator is a raised portion of the mask.

23. The kit of claim 22, wherein the skin mask is suitable for placement on the digit such that the raised portion of the locator points upwards away from the digit.

24. The kit of claim 20, wherein the nail treatment device locator comprises an insert or aperture into which the skin mask locator fits.

25. The kit of claim 20, wherein the skin mask is suitable for placement about a nail perimeter.

26. The kit of claim 20, wherein the skin mask locator is suitable for placement on the eponychium and the device is suitable for placement on the digit such that the nail treatment device locator corresponds and fits with the skin mask locator.

27. The kit of claim 20, wherein the device is less than 0.5 mm thick.

28. The kit of claim 20, wherein the device further comprises at least one power source.

29. The kit of claim 20, wherein the at least one counter electrode is configured in the same plane and around the sides of the at least one main active electrode.

30. The kit of claim 20, wherein the nail treatment device comprises an aperture suitable for application about a movement joint of the toe.

31. The kit of claim 20, wherein the skin mask material is impermeable to formulation.

32. The kit of claim 20, wherein the mask comprises a nail frame suitable for disposing on the area surrounding the nail.

33. The kit of claim 32, wherein the nail frame comprises polystyrene foam.

34. The kit of claim 20, wherein the skin mask locator facilitates placement of the nail treatment device on top of the skin mask, such that the at least one main active electrode does not contact the skin.

35. The kit of claim 20, wherein the nail treatment device is an iontophoressis delivery device.

36. The kit of claim 28, wherein the nail treatment device further comprises a device frame and wherein the at least one main electrode and the at least one counter electrode are electrically connected to the at least one power source and are disposed in spaced relation on the device frame.

37. A nail treatment device comprising:
an active delivery device for promoting delivery of an active agent into the nail to treat a nail infection;
a skin mask suitable for placement on a digit to cover the skin surrounding the nail; and
at least one locator means for accurate placement of the device onto the nail, the at least one locator means comprising:
a mask locator disposed on the skin mask and suitable for placement on the eponychium; and
a corresponding device locator disposed on the delivery device,
wherein the nail treatment device is suitable for placement on the digit such that the device locator corresponds and fits with the mask locator.

38. A method of treating a nail infection comprising:
applying a skin mask to skin surrounding a nail of a digit, such that the nail region is exposed, the skin mask comprising a skin mask locator;
positioning the skin mask locator about a position on the digit;
applying a formulation comprising an active agent to the nail;
applying a nail treatment device to the digit over the skin mask, the nail treatment device comprising at least one main electrode, at least one counter electrode and a device locator, such that the device locator interlocks with the skin mask locator and the formulation and the at least one main electrode are positioned on the nail;
wherein contacting the body area with the device causes closing of a circuit and current promotes delivery of the active agent from the formulation to the nail for treatment of the nail.

39. The method of claim 38, wherein the position on the digit comprises the eponychium.
